(19) United States
(12) Patent Application Publication
(10) Pub. No.: US 2024/0335603 A1
(43) Pub. Date: Oct. 10, 2024

(54) NASAL DELIVERY DEVICE AND ADAPTER-COUPLED SUCTION-TYPE NASAL DELIVERY KIT INCLUDING NASAL DELIVERY DEVICE

(71) Applicants: TOKO YAKUHIN KOGYO CO., LTD., Osaka-shi (JP); TAISEI KAKO CO., LTD., Osaka-shi, Osaka (JP)

(72) Inventors: Taizou KAMISHITA, Osaka-shi (JP); Takashi MIYAZAKI, Osaka-shi (JP); Tsuyoshi SOMEKAWA, Ibaraki-shi (JP); Yuki KATAGIRI, Ibaraki-shi (JP)

(21) Appl. No.: 18/425,717

(22) Filed: Jan. 29, 2024

(30) Foreign Application Priority Data
Apr. 4, 2023 (JP) 2023-060869

(51) Int. Cl. A61M 3/02

(52) U.S. Cl. CPC A61M 3/0279

(57) ABSTRACT

Provided is a nasal delivery device capable of easily delivering a medicinal solution. A nasal delivery device that delivers a medicinal solution from a vial enclosing the medicinal solution, the device comprising a nozzle portion that jets the medicinal solution, a syringe connected to the nozzle portion and having a substantially hollow cylindrical shape, a plunger disposed in an insertable manner in an axial direction between an initial position and a push-in position with respect to the syringe, the plunger including a main body having a substantially columnar shape, a gasket disposed at one end of the main body, and a disk portion that applies a pressing force, the disk portion being disposed at the other end of the main body, and a restoring elastic portion that deforms and generates a restoring force as the plunger moves from the initial position to the push-in position with respect to the syringe, wherein an inner wall of the syringe and the gasket of the plunger define a first space that holds the medicinal solution in the nasal delivery device, and the medicinal solution is delivered to the first space as the restoring elastic portion is restored to an original shape by the restoring force.

### TECHNICAL FIELD

The present disclosure relates to a nasal delivery device and an adapter-coupled suction-type nasal delivery kit including the nasal delivery device.

### BACKGROUND ART

Conventionally, as an injector type atomizer for administering a medicinal solution to a patient, for example, there is an injector type atomizer described in JP 2930526 B (Patent Document 1). The atomizer includes a cylinder having a jet nozzle at a tip, a piston fitted into the cylinder, and a rod having a tip attached to the piston and a rear end protruding more than a rear end of the cylinder. The rear end of the cylinder and the rear end of the rod are joined by a cushion material that squashes in stages according to a pressing force in an axial direction of the rod.

The medicinal solution is sealed and stored in an aseptic state in order to avoid contamination from various bacteria and the like, and the medicinal solution is filled in advance (or prefilled or delivered in advance) in a nasal delivery device on the day of administration.

### PATENT DOCUMENT (RELATED ART PATENT DOCUMENT)

### Patent Document

Patent Document 1: JP 2930526 B

### SUMMARY OF THE INVENTION

### Problems to be Solved by the Invention

As a result of intensive studies, the inventor of the present disclosure has noticed a problem that it takes time and effort of many personnel in a case where a medicinal solution is filled and prepared in advance on the day of administration. Even when a production facility is used, it has been found that it is necessary to provide a new production facility corresponding to the type of the medicinal solution, which requires a lot of time, labor, and cost. For this reason, there is a possibility that it is not possible to promptly cope with a pandemic or production of a small lot to be prepared for a test of a medicinal solution effect.

The present disclosure has been made in view of such problems. That is, an object of the present disclosure is to provide a nasal delivery device capable of easily delivering a medicinal solution. Another object of the present disclosure is to provide an adapter-coupled suction-type nasal delivery kit including such a nasal delivery device.

### Means for Solving the Problems

A nasal delivery device according to an embodiment of the present disclosure is a nasal delivery device that delivers a medicinal solution from a vial enclosing the medicinal solution, the device comprising:
a nozzle portion that jets the medicinal solution;
a syringe connected to the nozzle portion and having a substantially hollow cylindrical shape;
a plunger disposed in an insertable manner in an axial direction between an initial position and a push-in position with respect to the syringe, the plunger including a main body having a substantially columnar shape, a gasket disposed at one end of the main body, and a disk portion that applies a pressing force, the disk portion being disposed at the other end of the main body; and
a restoring elastic portion that deforms and generates a restoring force as the plunger moves from the initial position to the push-in position with respect to the syringe,
wherein an inner wall of the syringe and the gasket of the plunger define a first space that holds the medicinal solution in the nasal delivery device, and
the medicinal solution is delivered to the first space as the restoring elastic portion is restored to an original shape by the restoring force.

An adapter-coupled suction-type nasal delivery kit according to an embodiment of the present disclosure comprises:
the nasal delivery device described above; and
an adapter that engages with the nasal delivery device,
wherein the adapter includes:
   a first female engagement portion that engages with the nozzle portion;
   a second female engagement portion that faces the first female engagement portion and engages with the vial; and
   a needle including a first fluid flow path in fluid communication with the nozzle portion in a state where the nasal delivery device and the adapter are connected.

### Effects of the Invention

A nasal delivery device according to an embodiment of the present disclosure can easily deliver a medicinal solution. An adapter-coupled suction-type nasal delivery kit according to another embodiment of the present disclosure can easily deliver a medicinal solution to the nasal delivery device.

### BRIEF DESCRIPTION OF DRAWINGS

FIG. 1 is an exploded perspective view illustrating an adapter-coupled suction-type nasal delivery kit including a nasal delivery device according to a first embodiment;

FIG. 2 is a side view illustrating the nasal delivery device according to the first embodiment;

FIG. 3 is a side view illustrating an adapter-coupled suction-type nasal delivery kit according to a third embodiment;

FIG. 4 is a sectional view illustrating a restoring elastic portion of the nasal delivery device according to the first embodiment;

FIG. 5 is a sectional view illustrating an adapter of the adapter-coupled suction-type nasal delivery kit according to the third embodiment;

FIGS. 6A to 6C are side views illustrating a nasal delivery device when an adapter-coupled suction-type nasal delivery kit is used;

FIGS. 7A to 7C are perspective views for describing a method for using the adapter-coupled suction-type nasal delivery kit according to the third embodiment;

FIG. 8 is a perspective view for describing a method for using the nasal delivery device according to the first embodiment;

FIG. 9 is an exploded perspective view illustrating an adapter-coupled suction-type nasal delivery kit including a nasal delivery device according to a second embodiment; and

FIG. 10 is a sectional view illustrating an adapter in a nasal delivery kit according to a fifth embodiment.

### MODES FOR CARRYING OUT THE INVENTION

Hereinafter, a nasal delivery device and an adapter-coupled suction-type nasal delivery kit, which are embodiments of the present disclosure, will be described in detail with reference to the illustrated embodiments. The drawings include schematic drawings and may not reflect actual dimensions and ratios.

In the present specification, an adjective or a noun with the term “substantially” includes not only the strict meaning of the adjective or the noun with the term, but also an adjective or a noun within a range wherein actual variations are taken into consideration. For example, a substantially columnar shape includes not only a strict columnar shape but also a columnar shape in consideration of actual variations over time under manufacturing conditions.

Numerical ranges referred to herein are intended to also include lower and upper limits themselves, unless otherwise noted, such as “less than”, “larger than”, and “smaller than”. For example, taking a numerical range such as 1 to 10 as an example, the numerical range is interpreted as including the lower limit value “1” and the upper limit value “10”.

### First Embodiment: Nasal Delivery Device

A first embodiment relates to a nasal delivery device.

A nasal delivery device according to the first embodiment is a nasal delivery device that delivers a medicinal solution from a vial enclosing the medicinal solution, the device including:
a nozzle portion that jets the medicinal solution;
a syringe connected to the nozzle portion and having a substantially hollow cylindrical shape;
a plunger disposed in an insertable manner in an axial direction between an initial position and a push-in position with respect to the syringe, the plunger including a main body having a substantially columnar shape, a gasket disposed at one end of the main body, and a disk portion that applies a pressing force, the disk portion being disposed at the other end of the main body; and
a restoring elastic portion that deforms and generates a restoring force as the plunger moves from the initial position to the push-in position with respect to the syringe,
wherein an inner wall of the syringe and the gasket of the plunger define a first space that holds the medicinal solution in the nasal delivery device, and
the medicinal solution is delivered to the first space as the restoring elastic portion is restored to an original shape by the restoring force.

The nasal delivery device according to the first embodiment can easily deliver a medicinal solution. Without being bound by a particular theory, the reason is presumed as follows.

The nasal delivery device according to the first embodiment includes a restoring elastic portion that deforms and generates a restoring force with the movement of the plunger from the initial position to the push-in position with respect to the syringe. For this reason, for example, in the adapter-coupled suction-type nasal delivery kit according to a second embodiment connected to the adapter, when the plunger is pushed and moved from the initial position to the push-in position with respect to the syringe in a state of being connected to the vial, a restoring force is generated in the restoring elastic portion. Thus, only by releasing the pressing force applied to the plunger, the plunger is moved from the push-in position to the initial position by the restoring force, and the medicinal solution is filled in the first space. The nasal delivery device according to the first embodiment can thus easily deliver the medicinal solution.

### [Configuration of Nasal Delivery Device]

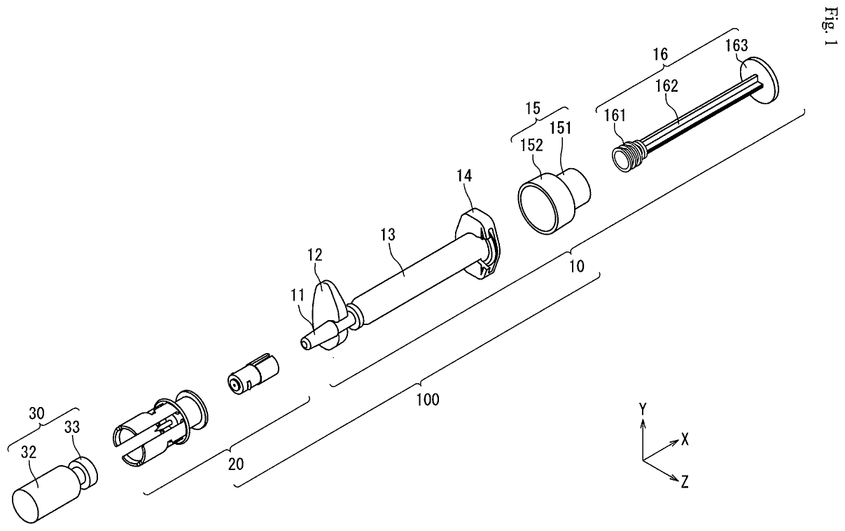

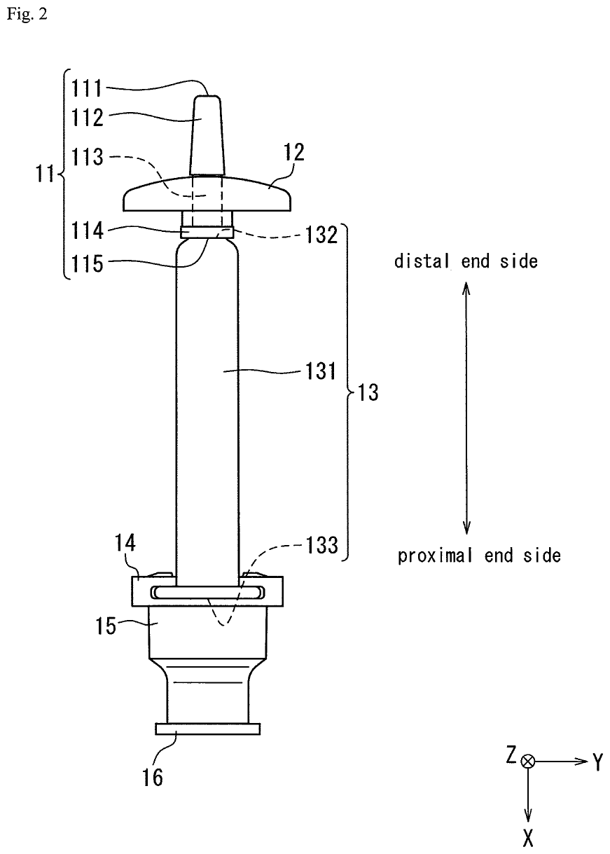

A configuration of the nasal delivery device according to the first embodiment will be described with reference to FIGS. 1 and 2. FIG. 1 is an exploded perspective view illustrating an adapter-coupled suction-type nasal delivery kit including the nasal delivery device according to the first embodiment. FIG. 2 is a side view illustrating the nasal delivery device according to the first embodiment.

In FIGS. 1 to 2, a moving direction (axial direction) of the plunger 16 in the syringe 13 is an X direction, a long axis direction of a nose rest 12 and a flange 14 is a Y direction in a direction orthogonal to the X direction, and a short axis direction of the nose rest 12 and the flange 14 is a Z direction in a direction orthogonal to the X direction.

As illustrated in FIGS. 1 and 2, a nasal delivery device 10 according to the first embodiment is a nasal delivery device that delivers a medicinal solution from a vial 30 (via an adapter 20) in which the medicinal solution is sealed.

The nasal delivery device 10 includes a nozzle portion 11, the syringe 13, a restoring elastic portion 15, the plunger 16, the nose rest 12, and the flange 14.

### (Nozzle Portion)

The nozzle portion 11 is disposed at a distal end (details will be described later) of the syringe 13 in fluid communication with a first opening 132 of the syringe 13 and sprays (jets) a medicinal solution (medicine). The nozzle portion 11 can discharge the medicinal solution in the form of microdroplets (for example, in the form of mist) to the outside from an opening 11a at a tip of the nozzle portion 11 by spraying (jetting). Since the nozzle portion 11 jets the medicinal solution in the form of microdroplets, the medicinal solution can be effectively administered into the body via a nasal cavity of a patient.

The nozzle portion 11 has a substantially hollow cylindrical shape. That is, the nozzle portion 11 has a cylindrical structure having a substantially columnar hollow portion and has two openings 111 and 115 at a position corresponding to a bottom surface (end surface) of the substantially columnar hollow portion. The two openings 111 and 115 are in fluid communication through the hollow portion. The opening 111 sprays (jets) the medicinal solution to the outside of the nasal delivery device 10. The opening 115 is in fluid communication with the first opening 132 of the syringe 13.

The nozzle portion 11 can be connected to an adapter 20 disposed between the vial 30 and the nasal delivery device 10, and a space communicating in the axial direction from a first space A to the inside of the vial 30 is sealed from the outside. Thus, moving the plunger 16 from the initial position to the push-in position can inject air into the vial 30 to increase the pressure in the vial when the nasal delivery device 10 is connected to the vial 30 via the adapter 20. The first space A is a space in the syringe 13 defined by the inner wall of the syringe and a gasket 161 of the plunger.

The nozzle portion 11 includes the opening 111, a conical portion 112, a constricted portion 113, and a flange portion 114. The opening 111 is disposed at a tip of the conical portion 112 and jets the medicinal solution to the outside of the nasal delivery device 10. The conical portion 112 has a substantially conical shape, has the opening 111 at a tip thereof, and is connected to the constricted portion 113 at a position axially facing the opening 111. The constricted portion 113 is connected to the conical portion 112, and the constricted portion 113 can detachably attach the nose rest 12. The diameter of the constricted portion 113 is smaller than the diameter of the conical portion 112 in a YZ sectional view. Since the diameter of the constricted portion 113 is smaller than those of the conical portion 112 and the flange portion 114 in the YZ sectional view, the nose rest 12 attached to the constricted portion 113 is prevented from moving in the axial direction. As a result, the nose rest 12 is stably fixed to the nozzle portion 11, which enables stable jetting of the medicinal solution into a nasal cavity. The diameter of the flange portion 114 is larger than the diameter of the conical portion 112 in the YZ sectional view.

The nozzle portion 11 is made of, for example, at least one material selected from the group consisting of a resin material, a glass material, a metal material, and a ceramic material.

### (Syringe)

The syringe 13 is connected to the nozzle portion 11 and has a substantially hollow cylindrical shape. The inner wall of the syringe 13 and the gasket 161 of the plunger 16 define the first space A that holds the medicinal solution in the nasal delivery device 10.

The syringe 13 includes a main body 131 having a substantially hollow cylindrical shape, first and second openings 132 and 133 disposed on both end sides in the axial direction of the main body 131, and a flange portion 134 disposed on a proximal end side of the main body 131.

The main body 131 holds the plunger 16 thereinside in an insertable manner. The first opening 132 is positioned at the distal end of the syringe 13 and is in fluid communication with the opening 115 of the nozzle portion 11. The medicinal solution is delivered to the first space A of the nasal delivery device 10 via the first opening 132. The second opening 133 is positioned at the proximal end of the syringe 13 and allows the plunger 16 to be inserted. The flange portion 134 has a diameter larger than that of the main body 131 in the YZ section and is connected to the flange 14.

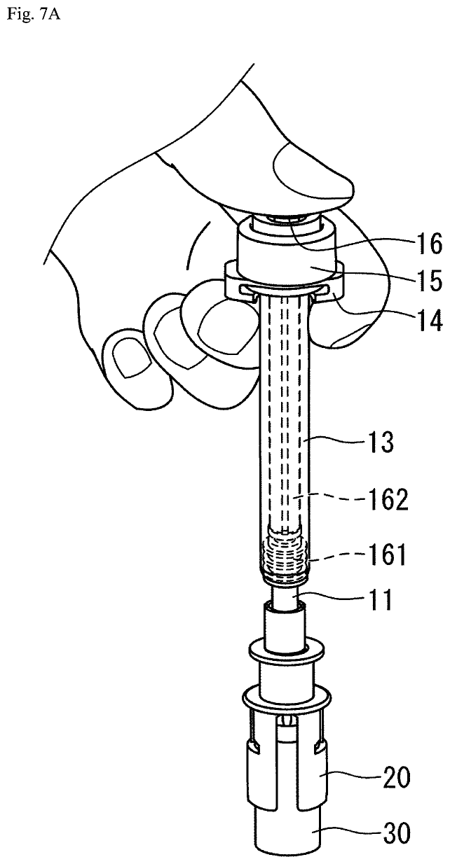

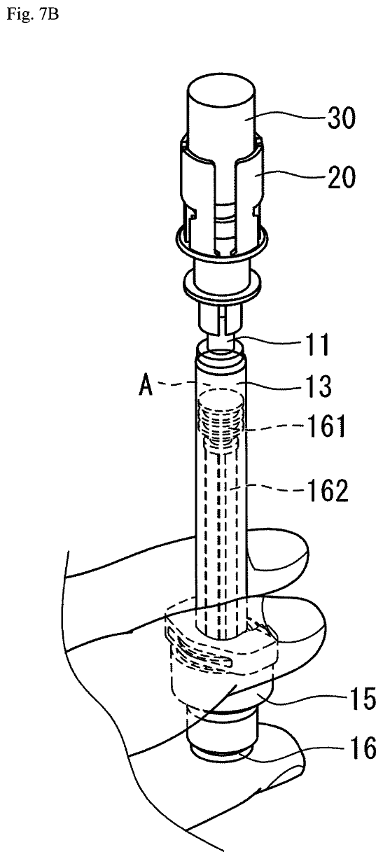

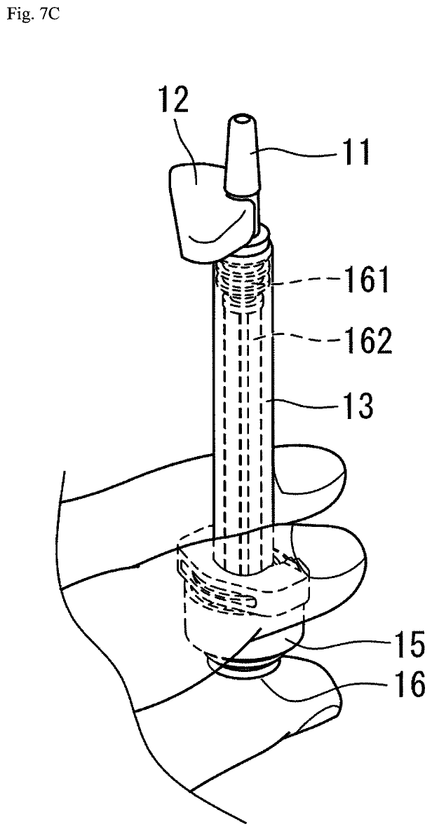

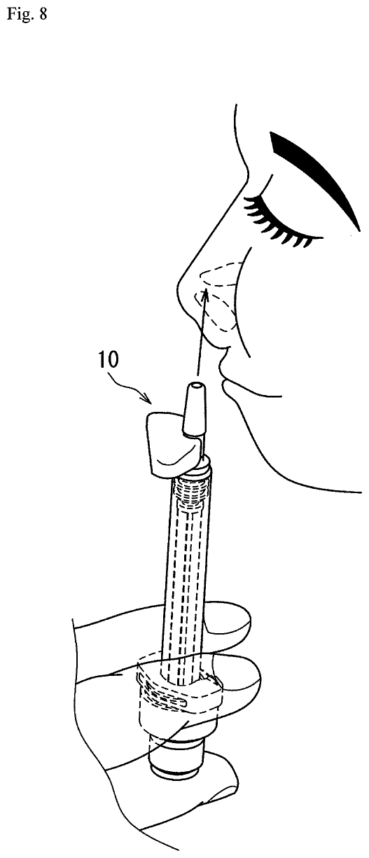

In the present specification, the proximal end corresponds to an open surface (open end) of the second opening 133 of the syringe 13 and refers to the end of the syringe 13 relatively closer to a user when the nasal delivery device 10 is used. The distal end corresponds to an opening surface (opening end) of the first opening 132 of the syringe 13 and refers to the end of the syringe 13 relatively far from the user when the nasal delivery device 10 is used. When using the nasal delivery device 10, the user hooks his/her finger on the flange 14 and a disk portion 163 of the plunger 16 as illustrated in FIGS. 7A to 7C and 8 to be described later.

In the present specification, in the X direction (axial direction) from the first opening 132 to the second opening 133 of the syringe 13, the proximal end side refers to a side on which the proximal end is relatively positioned, and the distal end side refers to a side on which the distal end is relatively positioned.

The syringe 13 is made of, for example, a transparent material (more specifically, at least one material selected from the group consisting of a transparent resin material and a transparent glass material). In the present specification, the term “transparent” refers to transparency to such an extent that the presence or absence of the medicinal solution 31 in the space A can be visually recognized when the nasal delivery device 10 is observed from the outside.

### (Flange)

The flange (or finger rest, finger grip, or finger hook portion) 14 is positioned near the proximal end of the syringe 13. Specifically, the flange 14 is disposed on the opening 133 side of the syringe 13 into which the plunger 16 is inserted. The flange 14 is connected with the flange portion 134 of the syringe 13. The flange 14 has a substantially elliptical shape in the YZ plane view. The flange 14 is a member on which a user hooks a finger when using the nasal delivery device 10, and more specifically, a member on which a user hooks a finger when delivering the medicinal solution 31 to the nasal delivery device 10 and when inserting the nozzle portion 11 of the nasal delivery device 10 into a nasal cavity. Since the nasal delivery device 10 includes the flange 14, it is possible to further improve the handling property at the time of delivering a medicinal solution and spraying the medicinal solution.

The flange 14 is made of, for example, at least one material selected from the group consisting of a resin material, a glass material, a metal material, and a ceramic material.

### (Plunger)

The plunger 16 is disposed in an insertable manner between the initial position and the push-in position with respect to the syringe 13. The movable range of the plunger 16 is the axial direction from the initial position to the push-in position. Since the disk portion 163 of the plunger 16 is connected to a first side surface portion 151 of the restoring elastic portion 15, the restoring elastic portion 15 deforms as the plunger 16 moves in the axial direction. More specifically, when the plunger 16 is positioned in the push-in position, the tip surface of the gasket 161 of the plunger 16 is brought into contact with the inner wall on the distal end side of the syringe 13. When the plunger 16 is positioned in the initial position, the restoring elastic portion 15 is in an initial state in which it is not deformed.

When the plunger 16 is moved from the initial position to the push-in position in a state where the nasal delivery device 10 is connected to the vial 30 via the adapter 20, air is pushed into the vial 30, and the pressure (internal pressure) in the vial 30 increases.

Then, when the plunger 16 is moved from the push-in position to the initial position, the medicinal solution is delivered from the vial 30 to the nasal delivery device 10. The plunger 16 is moved from the push-in position to the initial position by the restoring force of the restoring elastic portion 15. Thus, the nasal delivery device 10 can be easily and quickly filled.

When the plunger 16 is in the initial position, the first space A corresponds to the medicinal solution for one time of administration. Thus, by moving the plunger 16 from the push-in position to the initial position, the nasal delivery device 10 can accurately deliver the medicinal solution for one time of administration.

Next, removing the nasal delivery device 10 from the adapter 20, inserting the nozzle portion 11 into a nasal cavity, and moving the plunger 16 from the initial position to the push-in position in a state where the nasal delivery device 10 is filled with the medicinal solution causes the medicinal solution to be jetted from the nasal delivery device 10 into the nasal cavity.

In the present specification, the initial position refers to the position of the plunger 16 in a state where a force for pushing the plunger 16 is not applied. In the present specification, the push-in position refers to the position of the plunger 16 in a state where the tip of the plunger 16 (the tip of the gasket 161) is positioned at the distal end (open end) of the syringe 13 by applying a push-in force to the disk portion 163 of the plunger 16.

The plunger 16 includes a main body 162 having a substantially columnar shape, the gasket 161 disposed at one end of the main body 162, and a disk portion 163 disposed at the other end of the main body 162 and applying a pressing force. The main body 162 is connected to the gasket 161 at the distal end of the main body 162 and is connected to the disk portion 163 at the proximal end. The main body 162 has a substantially identical sectional shape (shape in the ZY cross section) with respect to the axial direction. That is, the main body 162 does not have a protrusion such as a rib. The term “substantially identical” includes not only strict identity but also realistic identity due to slight variation over time in manufacturing conditions. As illustrated in FIG. 1, the shape (sectional shape) of the main body 162 in the YZ section is a cross shape, but the shape is not limited to a cross shape, and it may be, for example, a substantially circle and a substantially ellipse. The main body 162 is made of at least one material selected from the group consisting of a resin material, a glass material, a metal material, and a ceramic material.

The gasket 161 is disposed at a distal end of the main body 162 having a substantially columnar shape. The gasket 161 is in close contact with the inner wall of the syringe 13 to increase airtightness of the first space A. The side surface of the gasket 161 is provided such that a first side surface portion having a relatively large diameter and a second side surface portion having a relatively small diameter in the YZ section are alternately arrayed in the axial direction. The gasket 161 has a plurality of first side surface portions and a plurality of second side surface portions. The plurality of first side surface portions abut the inner wall of the syringe 13. The plurality of first side surface portions abut the inner wall of the syringe 13 to prevent fluid communication between the first space A in the syringe 13 and the space on the main body 162 side of the plunger 16. This enhances airtightness of the first space A defined by the gasket 161 and the syringe 13. On the other hand, the second side surface portion does not abut the inner wall of the syringe 13. Appropriately reducing the contact area of the gasket 161 with the syringe 13 in this manner allows the plunger 16 to smoothly move in the axial direction in the syringe 13.

The gasket 161 has elasticity, and is made of, for example, a cushioning material (for example, a soft resin material and an elastomer material).

### (Restoring Elastic Portion)

The restoring elastic portion 15 is deformed with the movement of the plunger 16 from the initial position to the push-in position with respect to the syringe 13 to generate a restoring force. As the restoring elastic portion 15 is restored to its original shape by its restoring force, the medicinal solution is delivered to the first space A.

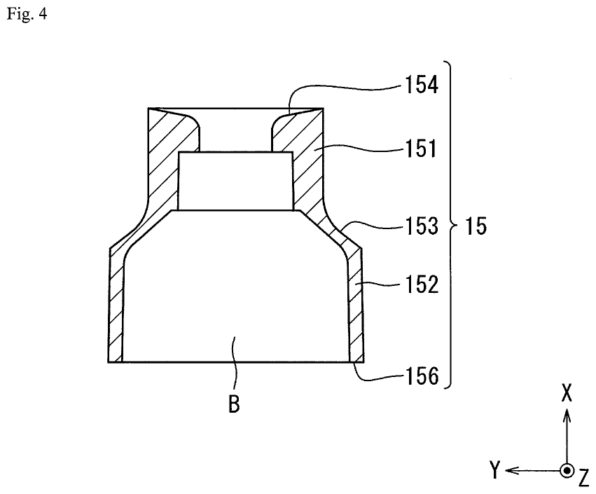

Further, the restoring elastic portion 15 will be described with reference to FIG. 4. FIG. 4 is a sectional view illustrating the restoring elastic portion 15 of the nasal delivery device 10. The restoring elastic portion 15 includes a first side surface portion 151 having a hollow cylindrical shape, a second side surface portion 152 having a hollow cylindrical shape and having a diameter larger than that of the first side surface portion 151, and an inclined portion 153 having a conical shape and connecting the first side surface portion 151 and the second side surface portion 152. That is, the restoring elastic portion 15 has a bellows shape having two different diameters via the inclined portion 153 in a section (ZX section) parallel to the moving direction of the plunger 16.

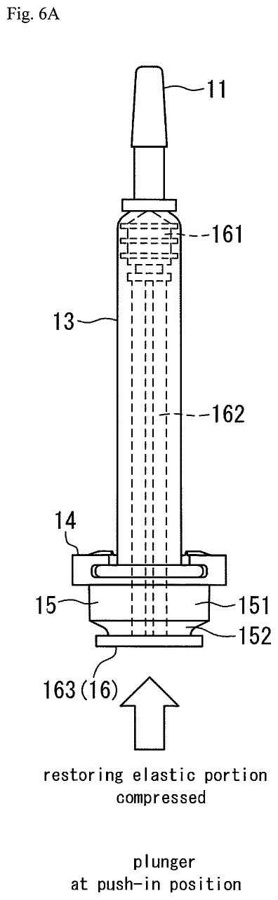

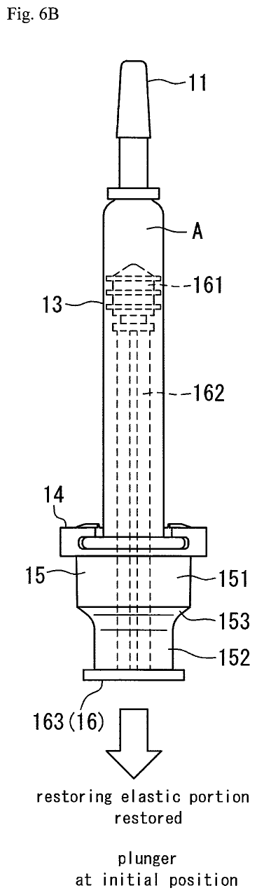

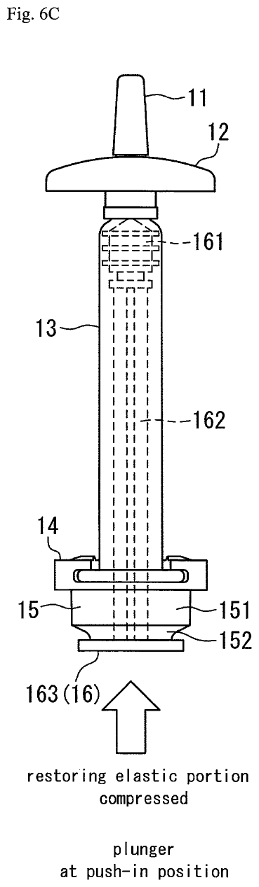

Since the restoring elastic portion 15 has the bellows structure, as the plunger 16 moves from the initial position to the push-in position with respect to the syringe 13, the first side surface portion 151 deforms in such a manner as to enter the inside of the second side surface portion 152 (in such a manner as to be folded). That is, the restoring elastic portion 15 deforms from the initial structure illustrated in FIG. 4 in which the plunger 16 is positioned at the initial position to the folding structure illustrated in FIG. 6A. Since the restoring elastic portion 15 deforms in one step in this manner, the nasal delivery device 10 administers the medicinal solution for one time of administration to a patient in one operation.

The restoring elastic portion 15 has a hollow structure. For example, the restoring elastic portion 15 does not have a support portion for deforming in two stages, and deforms in one stage as described above. In addition, since the disk portion 163 of the plunger 16 is connected to the first side surface portion 151, the restoring elastic portion 15 deforms along with the movement of the plunger 16 in the axial direction. Here, as illustrated in FIG. 4, the thicknesses of the first side surface portion 151 and the second side surface portion 152 are larger than the thickness of the inclined portion 153, and the inclined portion 153 is inclined in a single direction with respect to the axial direction. Thus, when stress is applied to the restoring elastic portion 15 by the movement of the plunger 16 from the initial position to the push-in position, mainly the inclined portion 153 of the restoring elastic portion 15 deforms. That is, in the XY section illustrated in FIG. 4, the restoring elastic portion 15 deforms so as not to generate a bending point in the inclined portion 153. More specifically, the deformation is performed such that the angle formed by (the outer surface of) the first side surface portion 151 and (the outer surface of) the inclined portion 153 decreases, and the angle formed by (the outer surface of) the second side surface portion 152 and (the outer surface of) the inclined portion 153 increases.

The restoring elastic portion 15 is made of, for example, a cushioning material (for example, a soft resin material and an elastomer material).

### (Nose Rest)

The nose rest 12 is detachably connected in such a manner as to sandwich the constricted portion 113 of the nozzle portion 11. FIG. 8 is a perspective view for describing a method for using the nasal delivery device 10. As illustrated in FIG. 8, the nozzle portion 11 can be inserted from an external nostril to a predetermined position in a nasal cavity by causing the nozzle portion 11 to abut against (into pressing contact with) the peripheral portion of the external nostril with the nose rest 12. Since the tip of the nozzle portion 11 can be adjusted to a desired direction in this manner, the medicinal solution 31 can be sprayed to a predetermined place in the nasal cavity, and the medicinal solution 31 can be effectively administered to the human body.

The nose rest 12 is made of, for example, at least one material selected from the group consisting of a resin material, a metal material, and a ceramic material.

### Second Embodiment: Nasal Delivery Device

A second embodiment relates to a nasal delivery device. A nasal delivery device according to the second embodiment is different from the nasal delivery device 10 according to the first embodiment in that a second space B defined by the restoring elastic portion, the disk portion of the plunger, and the flange is sealed from the outside. Hereinafter, this different configuration will be mainly described. In the second embodiment, the same reference numerals as those in the first embodiment have the same configurations as those in the first embodiment, and thus the description thereof will be omitted in principle.

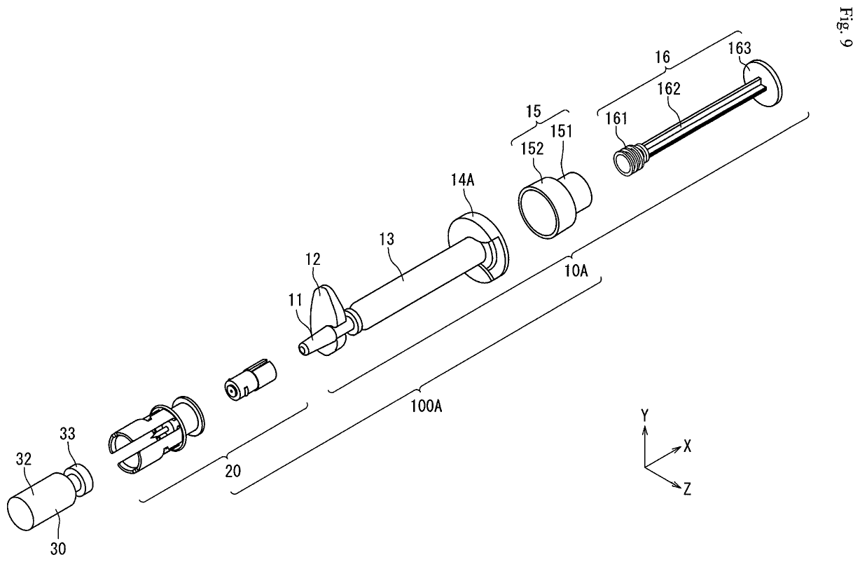

The nasal delivery device according to the second embodiment will be described with reference to FIG. 9. FIG. 9 is an exploded perspective view illustrating a nasal delivery device 10A according to the second embodiment. As illustrated in FIG. 9, a flange 14A has a substantially circular sectional shape in the YZ section. The diameter of the YZ section of the flange 14A is larger than the diameter of (the second side surface portion 152 of) the restoring elastic portion 15 in the YZ section. As described in the first embodiment, (the end surface 154 of) the first side surface portion 151 of the restoring elastic portion 15 is connected to the disk portion 163 of the plunger 16 by an adhesive or the like. (The end surface 156 of) the second side surface portion 152 of the restoring elastic portion 15 is connected to the flange 14A with an adhesive or the like. Thus, the second space B defined by the inner surface of the restoring elastic portion 15, the disk portion 163, and the flange 14A is sealed from the outside.

Since the second space B is sealed from the outside, the volume of the second space B decreases and the internal pressure of the second space B increases as the plunger 16 is moved from the initial position to the push-in position with respect to the syringe 13 by the pressing force applied to the disk portion 163.

Thus, eliminating the pressing force applied to the disk portion when the plunger 16 is in the push-in position causes the medicinal solution to be delivered from the vial 30 to the first space A due to the internal pressure when the nasal delivery device 10A is connected to the vial 30 via the adapter 20. That is, in the second embodiment, in a state where the nasal delivery device 10A is connected to the vial 30 via the adapter 20, the plunger 16 is moved from the push-in position to the initial position by the internal pressure of the second space B in addition to the internal pressure of the space communicating with the first space A and the restoring force of the restoring elastic portion 15 in the nasal delivery device 10A. With this configuration, in the nasal delivery device 10A according to the second embodiment, the medicinal solution is more easily delivered from the vial 30 to the nasal delivery device 10A.

### Third Embodiment: Adapter-Coupled Suction-Type Nasal Delivery Kit

A third embodiment relates to an adapter-coupled suction-type nasal delivery kit (hereinafter, it is also simply referred to as a “nasal delivery kit”) 100. The nasal delivery kit 100 according to the third embodiment is different from the nasal delivery device 10 according to the first embodiment in that it further includes the adapter 20. Hereinafter, this different configuration will be mainly described. In the third embodiment, the same reference numerals as those in the first embodiment have the same configurations as those in the first embodiment, and thus the description thereof will be omitted in principle.

As illustrated in FIG. 1, the nasal delivery kit 100 according to the third embodiment comprises:
the nasal delivery device 10 according to the first embodiment; and
an adapter 20 that engages with the nasal delivery device 10,
wherein the adapter 20 includes:
   a first female engagement portion 210 that engages with the nozzle portion 11;
   a second female engagement portion 220 that faces the first female engagement portion 210 and engages with the vial 30; and
   a needle 223 including a first fluid flow path 224 in fluid communication with the nozzle portion 11 in a state where the nasal delivery device 10 and the adapter 20 are connected.

The nasal delivery kit 100 according to the third embodiment can easily deliver the medicinal solution. Without being bound by a particular theory, the reason is presumed as follows.

The nasal delivery kit 100 according to the third embodiment includes the nasal delivery device 10 and the adapter 20 that engages with the nasal delivery device 10. The nasal delivery kit 100 can form a sealed system when the vial 30 in which a medicinal solution is enclosed is connected. Thus, when the plunger 16 is moved from the initial position to the push-in position in the nasal delivery device 10 to which the vial 30 is engaged, air (outside air) corresponding to the volume of the medicinal solution for one time of administration can be pushed (injected) into the vial 30. This causes the pressure (internal pressure) in the vial 30 to increase. When the pressing force applied to the plunger 16 is released, the plunger 16 moves from the push-in position to the initial position due to the internal pressure in addition to the restoring force of the restoring elastic portion 15 described in the first embodiment, and the medicinal solution 31 is filled in the first space A. Thus, the nasal delivery kit 100 according to the third embodiment can easily deliver the medicinal solution 31. In the present specification, the “outside air” refers to air present outside the sealed vial 30.

In the nasal delivery kit 100, the medicinal solution 31 can be delivered to the nasal delivery device 10 by moving the plunger 16 from the initial position to the push-in position with respect to the syringe 13 to inject the outside air into the vial 30 and increase the internal pressure in the vial 30, and moving the plunger 16 from the push-in position to the initial position with respect to the syringe 13 with the internal pressure and the restoring force of the restoring elastic portion 15 in a state where the nasal delivery device 10, the adapter 20, and the vial 30 are connected.

### (Factor Behind Present Disclosure)

The inventor of the present disclosure has paid attention to the fact that the nasal delivery kit 100 intranasally sprays the medicinal solution 31 to administer the medicinal solution to a patient. Normally, it should be avoided to introduce outside air into the vial 30 in which the medicinal solution is enclosed in an aseptic state as much as possible conventionally from the viewpoint of breaking the aseptic condition. It is then necessary to administer the medicinal solution to the patient in as short a time as possible from breaking of the aseptic condition of the vial 30. However, since various bacteria are present in a nasal cavity as a target of the medicinal solution administration, it has been found that some outside air can be introduced into the vial 30 enclosed in an aseptic state in the case of nasal administration for a short period of time. Based on such technical knowledge, the inventor has conceived the nasal delivery kit 100 connected to the vial 30 as a sealed system, in which the nasal delivery kit 100 delivers the medicinal solution 31 to the nasal delivery device 10 by injecting outside air into the vial 30 to increase the internal pressure in the vial 30, and using the restoring force of the restoring elastic portion 15 and the internal pressure in the vial 30 as a driving force.

### [Configuration of Nasal Delivery Kit]

### (Adapter)

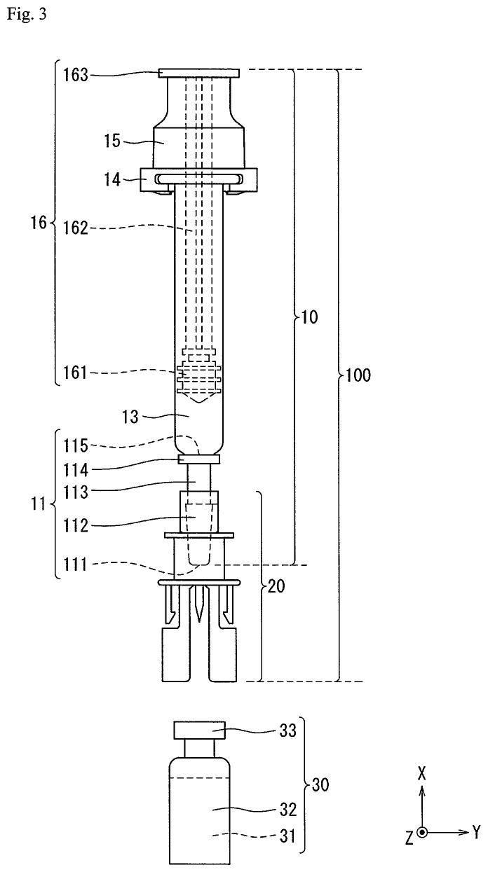

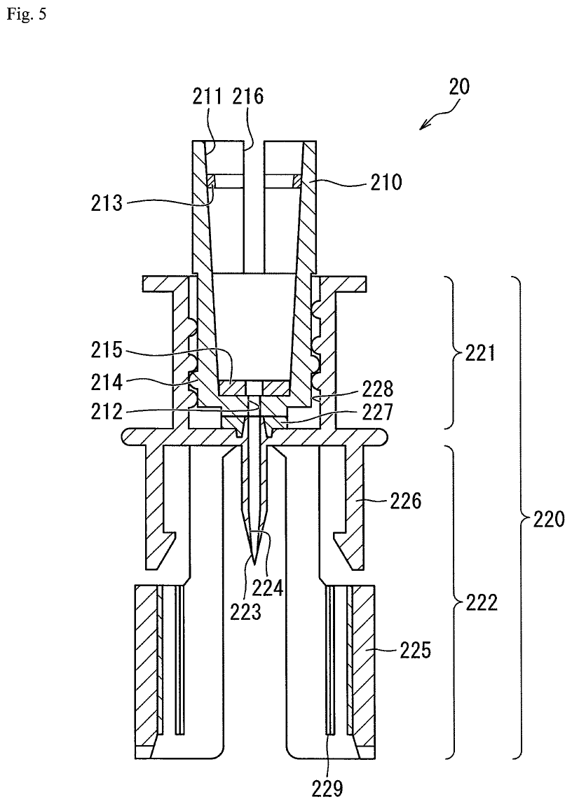

As illustrated in FIG. 3, the adapter 20 engages with the nasal delivery device 10. The adapter 20 will be further described with reference to FIG. 5. FIG. 5 is a sectional view illustrating the adapter 20 of the nasal delivery kit 100. As illustrated in FIG. 5, the adapter 20 includes a first female engagement portion 210 that engages with the nozzle portion 11, a second female engagement portion 220 that faces the first female engagement portion 210 in the axial direction and engages with the vial 30, and a needle 223 including a first fluid flow path 224 that is in fluid communication with the nozzle portion 11 in a state where the nasal delivery device 10 and the adapter 20 are connected. The adapter 20 includes two members of the first female engagement portion 210 and the second female engagement portion 220, and a male screw of the first female engagement portion 210 is fitted into a female screw of the second female engagement portion 220. The adapter 20 is made of, for example, at least one material selected from the group consisting of a resin material, a metal material, and a ceramic material except for elastic sealing portion 227 and 215.

### —First Female Engagement Portion—

The first female engagement portion 210 includes a recess 211 into which the nozzle portion 11 is fitted, a second fluid flow path 212 in fluid communication with the first fluid flow path 224 of the second female engagement portion 220, a rib 213 disposed on the second fluid flow path 212 side on the inner surface of the recess 211, a protrusion 214 disposed on a part of the outer surface, the elastic sealing portion 215, and a notch 216.

The protrusion 214 is disposed on a part of the outer surface of the first female engagement portion 210. The protrusion 214 is disposed in such a manner as to protrude to the outer surface of the first female engagement portion 210. That is, apart of the outer peripheral surface of the first female engagement portion 210 is a male screw, and the male screw is engaged with the female screw of the second female engagement portion 220.

The recess 211 allows the nozzle portion 11 to be fitted. The rib 213 is disposed on the opening side of the inner surface of the recess 211. The rib 213 has a ring shape in the YZ section. Thus, the tip of the nozzle portion 11 is fitted into the recess 211 in such a manner as to be surrounded by the ring-shaped rib 213 in the YZ section. With this configuration, the connection portion between the nasal delivery device 10 and the adapter 20 is excellent in sealability. When the first female engagement portion 210 has the rib 213 as described above, the adapter 20 is connected to the nasal delivery device 10 in such a manner s to sandwich the nozzle portion 11 with the rib 213. That is, the nasal delivery device 10 can be stably connected to the adapter 20. As a result, at the time of delivering the medicinal solution 31 to the nasal delivery device 10, the nasal delivery device 10 is hardly detached from the adapter 20. On the other hand, since the connection by the rib 213 is not strong, the nasal delivery device 10 can be easily removed from the adapter 20 after the delivery.

The elastic sealing portion 215 has a ring shape in the YZ section, whose opening is in fluid communication with the second fluid flow path 212. The elastic sealing portion 215 is, for example, an O-ring. The elastic sealing portion 215 is an elastic member having a cushioning property disposed on the bottom surface of the recess 211. The elastic sealing portion 215 is made of, for example, a cushioning material (for example, a rubber material, a soft resin material, and an elastomer material). Thus, the nasal delivery device 10 and the adapter 20 are connected between the tip of the nozzle portion 11 and the first female engagement portion 210 in such a manner as to squash the elastic sealing portion 215. When the nasal delivery device 10 and the adapter 20 are connected so as to squash the elastic sealing portion 215 at the connection portion like this, the sealability can be further improved. As a result, the sealability of the first space A is improved, and thus the internal pressure of the first space A can be easily used in the delivery of the medicinal solution 31 to the nasal delivery device 10.

There are two notches 216. The two notches 216 are disposed in such a manner as to face each other with an axis interposed therebetween in the YZ section. Thus, by inserting the planar member of each notch 216 and rotating the planar member about the axis, the first female engagement portion 210 and the second female engagement portion 220 can be tightened more tightly, and the sealability can be further enhanced. As a result, the sealability of the first space A is improved, and thus the internal pressure of the first space A can be easily used in the delivery of the medicinal solution 31 to the nasal delivery device 10.

### —Second Female Engagement Portion—

The second female engagement portion 220 includes a first recess 221 into which the nozzle portion 11 is fitted, a second recess 222 that connects the vial 30, the needle 223 that forms a hole in the vial 30 when the second female engagement portion 220 is connected to the vial 30, a hold portion 225 that holds a main body 32 of the vial 30, a claw portion 226 into which a lid portion 33 of the vial 30 is fitted, and the elastic sealing portion 227.

The inner surface of the first recess 221 is a female screw into which the second female engagement portion 220 is to be fitted. The elastic sealing portion 227 is disposed on the bottom surface of the first recess 221 and has an opening in fluid communication with the first fluid flow path 224. The elastic sealing portion 227 has a ring shape in the YZ section, whose opening is in fluid communication with the first fluid flow path 224. The elastic sealing portion 227 is, for example, an O-ring. The elastic sealing portion 227 is an elastic member having a cushioning property disposed on the bottom surface of the first recess 221. The elastic sealing portion 227 is made of, for example, a cushioning material (for example, a rubber material, a soft resin material, and an elastomer material). Thus, the first female engagement portion 210 and the second female engagement portion 220 are connected to each other in such a manner as to squash the elastic sealing portion 227 between the first female engagement portion 210 and the second female engagement portion 220. When the first female engagement portion 210 and the second female engagement portion 220 are connected in such a manner as to squash the elastic sealing portion 227 at the connection portion like this, the sealability can be further improved. As a result, the sealability of the first space A is improved, and thus the internal pressure of the first space A can be easily used in the delivery of the medicinal solution 31 to the nasal delivery device 10.

The hold portion 225 holds the main body 32 of the vial 30. The claw portion 226 allows the lid portion 33 of the vial 30 to fit. As described above, since the vial 30 is stably connected to the second female engagement portion 220 of the adapter 20 by the hold portion 225 and the claw portion 226, the connection portion between the vial 30 and the adapter 20 is fixed, and the sealability is easily maintained. As a result, a decrease in the sealability of the first space A is suppressed, and the internal pressure of the first space A can be easily used in the delivery of the medicinal solution 31 to the nasal delivery device 10.

A plurality of ribs 229 are disposed on the inner wall surface of the hold portion 225. Each rib 229 has a rectangular sectional shape. This plurality of ribs 229 is disposed along the axial direction and brought into contact with the outer surface of the vial 30 when the vial 30 is connected. That is, the hold portion 225 holds the vial 30 in such a manner as to be in contact with the plurality of ribs 229. With this configuration, rotation at the time of connecting the vial 30 is prevented, the connection between the vial 30 and the adapter 20 is strengthened, and the sealability of the first space A is enhanced.

### (Vial)

The vial 30 does not directly constitute the nasal delivery kit 100, but is an important matter in describing the nasal delivery kit 100, and thus it will be described.

The vial 30 encloses the medicinal solution 31 in an aseptic state. When the vial 30 is connected to the nasal delivery kit 100, the vial 30 is engaged with the second female engagement portion 220 of the adapter 20 in the nasal delivery kit 100. At this time, the needle 223 of the adapter 20 forms a hole on the top surface of (the lid portion 33 of) the vial 30. Since the needle 223 has the first fluid flow path 224 in fluid communication with the nozzle portion 11, the inside of the vial 30 and the inside of the syringe 13 are in fluid communication with each other in a state where the vial 30 is connected to the nasal delivery kit 100.

Since the restoring force of the restoring elastic portion 15 and the internal pressure of the vial 30 are used as the driving force, the medicinal solution 31 can be delivered even when its viscosity is relatively high. The viscosity of such a medicinal solution 31 is, for example, 100 to 5,000 mPa·s at 25° C.

Since the nasal delivery kit 100 is for nasal administration, the medicinal solution 31 can be easily delivered to the nasal delivery device 10 even when the medicinal solution 31 is a vaccine.

[Method for Using Nasal Device: Delivery to Nasal Delivery Device and Spray of Medicinal Solution into Nasal Cavity]

An example of a method for using a nasal device will be described with reference to FIGS. 6A to 6C to 8. FIGS. 6A to 6C are side views illustrating a nasal delivery device when the nasal device is used. FIGS. 7A to 7C are perspective views for describing a method for using the nasal delivery kit 100.

As illustrated in FIG. 7A, in the nasal delivery kit 100 connected to the vial 30, the plunger 16 is pushed and moved from the initial position to the push-in position in a state where the nasal delivery kit 100 is disposed such that the vial 30 is in the vertical direction. Here, the nose rest 12 is removed from the nasal delivery kit 100. With this configuration, air corresponding to the volume of the medicinal solution for one time of administration is pushed into the vial 30, and the pressure (internal pressure) in the vial 30 increases. At this time, as illustrated in FIG. 6A, in the nasal delivery device 10, the restoring elastic portion 15 is folded such that the first side surface portion 151 having a relatively small diameter enters the inside of the second side surface portion 152 having a relatively large diameter. A force (restoring force) to return to the original shape is generated in the restoring elastic portion 15.

As illustrated in FIG. 7B, the force applied to the plunger 16 is released in a state where the nasal delivery kit 100 is disposed such that the vial 30 is in the reverse vertical direction. Here, the plunger 16 moves from the push-in position to the initial position due to the restoring force of the restoring elastic portion 15 and the internal pressure of the vial 30. This causes the medicinal solution 31 to be delivered to the nasal delivery device 10 and held in the first space A. Since the medicinal solution 31 is delivered such that the vial 30 is in the reverse vertical direction, air in the vial 30 is not delivered to the nasal delivery device 10. At this time, as illustrated in FIG. 6B, in the nasal delivery device 10, the plunger 16 moves from the push-in position to the initial position in such a manner that the restoring elastic portion 15 returns to the original shape.

As illustrated in FIG. 7C, the nose rest 12 is attached to the nasal delivery device 10, and the opening of the nozzle portion 11 is disposed in such a manner as to face the reverse vertical direction, and a part of the nozzle portion 11 is inserted into a nasal cavity. In this state, as illustrated in FIG. 6C, the plunger 16 is moved from the initial position to the push-in position, and the medicinal solution 31 is sprayed into the nasal cavity.

### Fourth Embodiment: Nasal Delivery Kit

A fourth embodiment relates to a nasal delivery kit. The nasal delivery kit according to the fourth embodiment is different from the nasal delivery kit 100 according to the third embodiment in that the nasal delivery device 10A is provided instead of the nasal delivery device 10. Hereinafter, this different configuration will be mainly described. In the fourth embodiment, the same reference numerals as those in the first to third embodiments have the same configurations as those in the first to third embodiments, and thus the description thereof will be omitted in principle.

In the nasal delivery device 10A in a nasal delivery kit 100A according to the fourth embodiment, the second space B defined by the inner surface of the restoring elastic portion 15, the disk portion 163 of the plunger 16, and the flange 14 is sealed from the outside. Thus, as illustrated in FIG. 9, the medicinal solution 31 can be delivered to the nasal delivery device 10A by moving the plunger 16 from the initial position to the push-in position with respect to the flange 14 to increase the internal pressure of the second space B, and moving the plunger 16 from the push-in position to the initial position with respect to the syringe 13 with the internal pressure in a state where the nasal delivery device 10A, the adapter 20, and the vial 30 are connected.

That is, in the nasal delivery kit 100A according to the fourth embodiment, the medicinal solution 31 can be delivered to the nasal delivery device 10A using the internal pressure of the second space B in addition to the internal pressure of the first space A and the restoring force of the restoring elastic portion 15 in the third embodiment. Thus, the nasal delivery kit 100A can deliver the medicinal solution 31 to the nasal delivery device 10A more easily.

### Fifth Embodiment: Nasal Delivery Kit

A fifth embodiment relates to a nasal delivery kit. The nasal delivery kit according to the fifth embodiment is different from the nasal delivery kit 100 according to the third embodiment in that an adapter 20B is provided instead of the adapter 20. Hereinafter, this different configuration will be mainly described. In the fourth embodiment, the same reference numerals as those in the first to third embodiments have the same configurations as those in the first to third embodiments, and thus the description thereof will be omitted in principle.

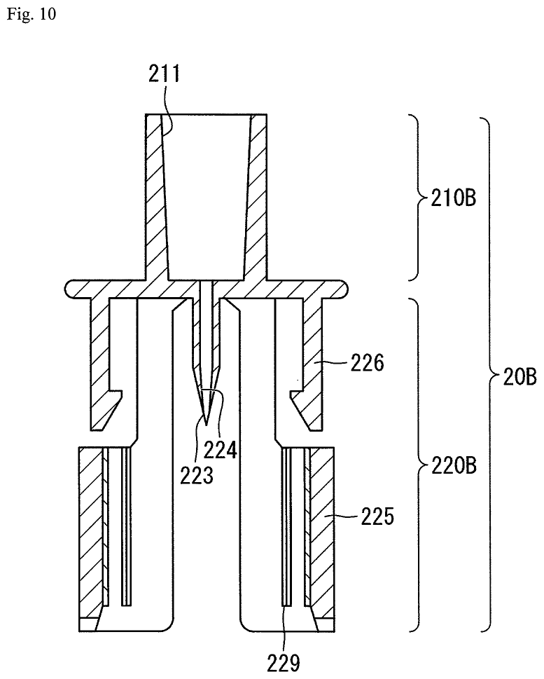

With reference to FIGS. 9 and 10, an adapter in the nasal delivery kit according to the fifth embodiment will be described. FIG. 10 is a sectional view illustrating an adapter in the nasal delivery kit according to a fifth embodiment. The adapter 20B is different from the adapter 20 according to the fourth embodiment in that a first female engagement portion 210B and a second female engagement portion 220B are integrated. Strictly speaking, the adapter 20B is different from the adapter 20 in that the adapter 20B does not further include the elastic sealing portion 215 or the rib 213. However, in another aspect, the adapter 20B may have at least one of these.

In the adapter 20B, since there are few joint portions in the adapter 20B, the sealability of the space communicating in the axial direction from the first space A to the inside of the vial 30 is further improved in a state where the nasal delivery device 10, the adapter 20B, and the vial 30 are connected. With this configuration, the internal pressure is likely to increase, and the medicinal solution 31 can be delivered to the nasal delivery device 10A.

### OTHER EMBODIMENTS

The present disclosure is not limited to the above-described embodiments and can be modified in design without departing from the gist of the present disclosure.

In the first embodiment, the flange 14 has a substantially elliptical shape in a YZ plane view, but the flange 14 is not limited to this configuration. For example, the flange 14 may take any shape as long as a finger and the restoring elastic portion can be hooked on the flange 14.

The rib 213 is disposed on the opening side of the inner surface of the recess 211, but the rib is not limited to this configuration. The rib 213 may be disposed on the second fluid flow path 212 side of the inner surface of the recess 211.

Aspects of the nasal delivery device and the nasal delivery kit including the nasal delivery device according to the present disclosure are as follows.

<1>

A nasal delivery device that delivers a medicinal solution from a vial enclosing the medicinal solution, the device comprising:
a nozzle portion that jets the medicinal solution;
a syringe connected to the nozzle portion and having a substantially hollow cylindrical shape;
a plunger disposed in an insertable manner in an axial direction between an initial position and a push-in position with respect to the syringe, the plunger including a main body having a substantially columnar shape, a gasket disposed at one end of the main body, and a disk portion that applies a pressing force, the disk portion being disposed at the other end of the main body; and
a restoring elastic portion that deforms and generates a restoring force as the plunger moves from the initial position to the push-in position with respect to the syringe,
wherein an inner wall of the syringe and the gasket of the plunger define a first space that holds the medicinal solution in the nasal delivery device, and
the medicinal solution is delivered to the first space as the restoring elastic portion is restored to an original shape by the restoring force.

<2>

The nasal delivery device according to <1>, wherein
the nozzle portion is connectable with an adapter disposed between the vial and the nasal delivery device, and
a space communicating in the axial direction from the first space to an inside of the vial is sealed from an outside.

<3>

The nasal delivery device according to <1> or <2>, wherein the restoring elastic portion deforms in one step.

<4>

The nasal delivery device according to any one of <1> to <3>, wherein the restoring elastic portion has a hollow structure.

<5>

The nasal delivery device according to any one of <1> to <4>, wherein the restoring elastic portion includes a first side surface portion having a hollow cylindrical shape, a second side surface portion having a hollow cylindrical shape and having a larger diameter than the first side surface portion, and an inclined portion having a conical shape and connecting the first side surface portion and the second side surface portion.

<6>

The nasal delivery device according to <5>, wherein the first side surface portion deforms in such a manner as to enter an inside of the second side surface portion as the plunger moves from the initial position to the push-in position with respect to the syringe.

<7>

The nasal delivery device according to <5> or <6>, the device further including a flange on an opening side of the syringe into which the plunger is to be inserted,
wherein the first side surface portion of the restoring elastic portion is connected to the flange,
the second side surface portion of the restoring elastic portion is connected to the disk portion of the plunger, and
a second space defined by an inner surface of the restoring elastic portion, the disk portion, and the flange is sealed from an outside.

<8>

The nasal delivery device according to <7>, wherein a volume of the second space decreases and an internal pressure of the second space increases as the plunger is moved from the initial position to the push-in position with respect to the syringe by a pressing force applied to the disk portion.

<9>

The nasal delivery device according to <7> or <8>, wherein the restoring elastic portion deforms with a movement of the plunger in the axial direction.

<10>

The nasal delivery device according to any one of <5> to <9>, wherein thicknesses of the first side surface portion and the second side surface portion are larger than a thickness of the inclined portion.

<11>

The nasal delivery device according to any one of <5> to <10>, wherein the inclined portion is inclined in a single direction with respect to the axial direction.

<12>

The nasal delivery device according to any one of <1> to <11>, wherein the plunger has a substantially identical sectional shape with respect to the axial direction.

<13>

The nasal delivery device according to any one of <1> to <12>, wherein when the plunger is in the initial position, the first space corresponds to the medicinal solution for one time of administration.

<14>

An adapter-coupled suction-type nasal delivery kit comprising:
the nasal delivery device according to any one of <1> to <13>; and
an adapter that engages with the nasal delivery device,
wherein the adapter includes:
   a first female engagement portion that engages with the nozzle portion;
   a second female engagement portion that faces the first female engagement portion and engages with the vial; and
   a needle including a first fluid flow path in fluid communication with the nozzle portion in a state where the nasal delivery device and the adapter are connected.

<15>

The adapter-coupled suction-type nasal delivery kit according to <14>, wherein
the first female engagement portion further includes an elastic sealing portion, and
the nasal delivery device and the adapter connect with each other between a tip of the nozzle portion and the first female engagement portion in such a manner as to squash the elastic sealing portion.

<16>

The adapter-coupled suction-type nasal delivery kit according to <14> or <15>, wherein
the first female engagement portion further includes a rib on an inner surface of a recess in a sectional view, and
the adapter connects with the nasal delivery device in such a manner as to sandwich the nozzle portion with the rib.

<17>

The adapter-coupled suction-type nasal delivery kit according to any one of <14> to <16>, wherein
the nasal delivery device further includes a flange on an opening side of the syringe into which the plunger is to be inserted, and
in a state where the nasal delivery device, the adapter, and the vial are connected, the plunger is moved from the initial position to the push-in position with respect to the flange to inject outside air into the vial and increase an internal pressure in the vial, and the plunger is moved from the push-in position to the initial position with respect to the syringe by the internal pressure and a restoring force of the restoring elastic portion to deliver the medicinal solution to the delivery device.

<18>

The adapter-coupled suction-type nasal delivery kit according to <17>, wherein
the nasal delivery device further includes a flange on an opening side of the syringe into which the plunger is to be inserted,
the restoring elastic portion includes a first side surface portion having a hollow cylindrical shape, a second side surface portion having a hollow cylindrical shape and having a larger diameter than the first side surface portion, and an inclined portion having a conical shape and connecting the first side surface portion and the second side surface portion,
the first side surface portion of the restoring elastic portion is connected to the flange,
the second side surface portion of the restoring elastic portion is connected to the disk portion of the plunger,
a second space defined by an inner surface of the restoring elastic portion, the disk portion, and the flange is sealed from an outside, and
in a state where the nasal delivery device, the adapter, and the vial are connected, the plunger is moved from the initial position to the push-in position with respect to the flange to increase an internal pressure of the second space, and the plunger is moved from the push-in position to the initial position with respect to the syringe by the internal pressure to deliver the medicinal solution to the delivery device.

<19>

The adapter-coupled suction-type nasal delivery kit according to any one of <14> to <18>, wherein the medicinal solution is a vaccine.

<20>

The adapter-coupled suction-type nasal delivery kit according to any one of <14> to <19>, wherein the medicinal solution has a viscosity of 100 to 5,000 mPa·s at 20° C.

<21>

The adapter-coupled suction-type nasal delivery kit according to any one of <14> to <20>, wherein the first female engagement portion and the second female engagement portion are integrated.

### INDUSTRIAL APPLICABILITY

The nasal delivery device and the adapter-coupled suction-type nasal delivery kit according to the present disclosure can be used to deliver a medicinal solution and administer the medicinal solution to a patient through the nose.

1. A nasal delivery device for delivering a medicinal solution from a vial enclosing the medicinal solution, the device comprising:
a nozzle portion that jets the medicinal solution;
a syringe connected to the nozzle portion and having a substantially hollow cylindrical shape;
a plunger disposed in an insertable manner in an axial direction between an initial position and a push-in position with respect to the syringe, the plunger including a main body having a substantially columnar shape, a gasket disposed at one end of the main body, and a disk portion that applies a pressing force, the disk portion being disposed at the other end of the main body; and
a restoring elastic portion that deforms and generates a restoring force as the plunger moves from the initial position to the push-in position with respect to the syringe,
wherein an inner wall of the syringe and the gasket of the plunger define a first space that holds the medicinal solution in the nasal delivery device, and
the medicinal solution is delivered to the first space as the restoring elastic portion is restored to an original shape by the restoring force.

2. The nasal delivery device according to claim 1, wherein
the nozzle portion is connectable with an adapter disposed between the vial and the nasal delivery device, and
a space communicating in the axial direction from the first space to an inside of the vial is sealed from an outside.

3. The nasal delivery device according to claim 1, wherein the restoring elastic portion deforms in one step.

4. The nasal delivery device according to claim 1, wherein the restoring elastic portion has a hollow structure.

5. The nasal delivery device according to claim 1, wherein the restoring elastic portion includes a first side surface portion having a hollow cylindrical shape, a second side surface portion having a hollow cylindrical shape and having a larger diameter than the first side surface portion, and an inclined portion having a conical shape and connecting the first side surface portion and the second side surface portion.

6. The nasal delivery device according to claim 5, wherein the first side surface portion deforms in such a manner as to enter an inside of the second side surface portion as the plunger moves from the initial position to the push-in position with respect to the syringe.

7. The nasal delivery device according to claim 5, the device further including a flange on an opening side of the syringe into which the plunger is to be inserted, wherein
the first side surface portion of the restoring elastic portion is connected to the flange,
the second side surface portion of the restoring elastic portion is connected to the disk portion of the plunger, and
a second space defined by an inner surface of the restoring elastic portion, the disk portion, and the flange is sealed from an outside.

8. The nasal delivery device according to claim 7, wherein a volume of the second space decreases and an internal pressure of the second space increases as the plunger is moved from the initial position to the push-in position with respect to the syringe by a pressing force applied to the disk portion.

9. The nasal delivery device according to claim 7, wherein the restoring elastic portion deforms with a movement of the plunger in the axial direction.

10. The nasal delivery device according to claim 5, wherein thicknesses of the first side surface portion and the second side surface portion are larger than a thickness of the inclined portion.

11. The nasal delivery device according to claim 5, wherein the inclined portion is inclined in a single direction with respect to the axial direction.

12. The nasal delivery device according to claim 1, wherein the plunger has a substantially identical sectional shape with respect to the axial direction.

13. The nasal delivery device according to claim 1, wherein when the plunger is in the initial position, the first space corresponds to the medicinal solution for one time of administration.

14. An adapter-coupled suction-type nasal delivery kit comprising:
the nasal delivery device according to claim 1; and
an adapter that engages with the nasal delivery device,
wherein the adapter includes:
a first female engagement portion that engages with the nozzle portion;
a second female engagement portion that faces the first female engagement portion and engages with the vial; and
a needle including a first fluid flow path in fluid communication with the nozzle portion in a state where the nasal delivery device and the adapter are connected.

15. The adapter-coupled suction-type nasal delivery kit according to claim 14, wherein
the first female engagement portion further includes an elastic sealing portion, and
the nasal delivery device and the adapter connect with each other between a tip of the nozzle portion and the first female engagement portion in such a manner as to squash the elastic sealing portion.

16. The adapter-coupled suction-type nasal delivery kit according to claim 14, wherein
the first female engagement portion further includes a rib on an inner surface of a recess in a sectional view, and
the adapter connects with the nasal delivery device in such a manner as to sandwich the nozzle portion with the rib.

17. The adapter-coupled suction-type nasal delivery kit according to claim 14, wherein
the nasal delivery device further includes a flange on an opening side of the syringe into which the plunger is to be inserted, and
in a state where the nasal delivery device, the adapter, and the vial are connected, the plunger is moved from the initial position to the push-in position with respect to the flange to inject outside air into the vial and increase an internal pressure in the vial, and the plunger is moved from the push-in position to the initial position with respect to the syringe by the internal pressure and a restoring force of the restoring elastic portion to deliver the medicinal solution to the delivery device.

18. The adapter-coupled suction-type nasal delivery kit according to claim 17, wherein
the nasal delivery device further includes a flange on an opening side of the syringe into which the plunger is to be inserted,
the restoring elastic portion includes a first side surface portion having a hollow cylindrical shape, a second side surface portion having a hollow cylindrical shape and having a larger diameter than the first side surface portion, and an inclined portion having a conical shape and connecting the first side surface portion and the second side surface portion,
the first side surface portion of the restoring elastic portion is connected to the flange,
the second side surface portion of the restoring elastic portion is connected to the disk portion of the plunger,
a second space defined by an inner surface of the restoring elastic portion, the disk portion, and the flange is sealed from an outside, and
in a state where the nasal delivery device, the adapter, and the vial are connected, the plunger is moved from the initial position to the push-in position with respect to the flange to increase an internal pressure of the second space, and the plunger is moved from the push-in position to the initial position with respect to the syringe by the internal pressure to deliver the medicinal solution to the delivery device.

19. The adapter-coupled suction-type nasal delivery kit according to claim 14, wherein the medicinal solution is a vaccine.

20. The adapter-coupled suction-type nasal delivery kit according to claim 14, wherein the medicinal solution has a viscosity of 100 to 5,000 mPa·s at 20° C.

21. The adapter-coupled suction-type nasal delivery kit according to claim 14, wherein the first female engagement portion and the second female engagement portion are integrated.
